(19) United States
(12) Patent Application Publication
(10) Pub. No.: US 2025/0006374 A1
(43) Pub. Date: Jan. 2, 2025

(54) METHOD, SYSTEM, APPARATUS, AND PROGRAM FOR DETERMINING DEGREE OF POSSIBILITY OF SECONDARY HYPERTENSION

(71) Applicant: CureApp, Inc., Tokyo (JP)

(72) Inventor: Shin Suzuki, Tokyo (JP)

(73) Assignee: CureApp, Inc., Tokyo (JP)

(21) Appl. No.: 18/757,890

(22) Filed: Jun. 28, 2024

(30) Foreign Application Priority Data
Jun. 30, 2023 (JP) 2023-107810

(51) Int. Cl. G16H 50/20; A61B 5/021

(52) U.S. Cl. CPC G16H 50/20; A61B 5/021

(57) ABSTRACT

A method for determining a degree of possibility of secondary hypertension includes causing one or more computers to execute a step of acquiring user-related data including blood pressure value data indicating a blood pressure value measured and behavior record data indicating behavior performed by a user and a step of determining, based on the user-related data acquired, an essential hypertension prediction model based on a behavior score indicating the behavior of the user, and a secondary hypertension prediction model, degrees of possibilities of essential hypertension and secondary hypertension.

### TECHNICAL FIELD

The present invention relates to a method, system, apparatus and program for determining a degree of possibility of secondary hypertension.

### BACKGROUND ART

There are two types of hypertension: essential hypertension and secondary hypertension. The essential hypertension is hypertension having no underlying disease causing hypertension and unknown cause. It is known that various factors such as meals and lifestyle habits are involved in the cause of essential hypertension. On the other hand, secondary hypertension is hypertension caused by a disease of a thyroid gland, adrenal gland, or the like.

Regarding essential hypertension, there is a therapeutic app that can support therapy of essential hypertension by being installed on a smartphone. A patient, using the therapeutic app, inputs a blood pressure measurement result at home (home blood pressure value) and modifies behavior to continuously improve meals and lifestyle habits, thereby obtaining an effect of lowering a blood pressure (Non-Patent Document 1).

### CITATION LIST

### Non-Patent Document

Non-Patent Document 1: CureApp HT, Product summary site of a Prescription Digital Therapeutic App for Hypertension (for patients) (URL: https://cureapp.co.jp/productsite/ht/forpatient/)

### SUMMARY OF THE INVENTION

### Technical Problem

When using the above-described therapeutic app, a patient with secondary hypertension may continuously use the therapeutic app without considering the possibility of secondary hypertension even though a sufficient effect of lowering a blood pressure cannot be obtained. There has not been achieved a system for determining a degree of possibility of secondary hypertension for a patient whose blood pressure is not lowered even though behavior for lowering the blood pressure is performed.

### Means for Solving the Problem

1. The present invention has been made in view of the problems described above and has the following features. That is, a method according to an aspect of the present invention is a method for determining a degree of possibility of secondary hypertension. The method includes causing one or more computers to execute a step of acquiring user-related data including blood pressure value data indicating a blood pressure value measured and behavior record data indicating behavior performed by a user and a step of determining, based on the user-related data acquired, an essential hypertension prediction model based on a behavior score indicating the behavior of the user, and a secondary hypertension prediction model, degrees of possibilities of essential hypertension and secondary hypertension.
2. In the method according to 1, the step of determining degrees of possibilities of essential hypertension and secondary hypertension may include calculating, based on the acquired user-related data, an error term in the essential hypertension prediction model and an error term in the secondary hypertension prediction model, a step of calculating, based on the calculated error term in the essential hypertension prediction model, likelihood when hypertension is assumed to be essential hypertension, a step of calculating, based on the calculated error term in the secondary hypertension prediction model, likelihood when hypertension is assumed to be secondary hypertension, and a step of determining, based on the calculated likelihood when hypertension is assumed to be essential hypertension and the calculated likelihood when hypertension is assumed to be secondary hypertension, the degrees of possibilities of essential hypertension and secondary hypertension.
3. The method according to 1 or 2 may include a step of determining a degree of possibility of secondary hypertension compared with the determined possibility of essential hypertension.
4. The method according to 3 may further include a step of presenting, upon input of request operation to a terminal of the user or a medical service worker, information indicating the degree of possibility of secondary hypertension compared with the determined possibility of essential hypertension on the terminal.
5. In the method according to any one of 1 to 4, the essential hypertension prediction model may include a term related to the behavior score, and the secondary hypertension prediction model may include no term related to the behavior score.
6. In the method according to any one of 1 to 5, the behavior record data may include at least one of an amount of exercise, a weight, and an amount of salt intake of the user.
7. In the method according to any one of 1 to 6, the user-related data may be acquired in a predetermined time period and may include two or more pieces of the blood pressure value data and two or more pieces of the behavior record data.
8. In the method according to any one of 1 to 7, the user-related data may include information indicating the blood pressure value and the behavior performed by the user and may be transmitted by a terminal of the user, the information being input by the user to the terminal of the user.
9. A computer program according to an aspect of the present invention causes one or more computers to execute the method according to any one of 1 to 8.
10. A system according to an aspect of the present invention is a system for determining a degree of possibility of secondary hypertension. The system acquires user-related data including blood pressure value data indicating a blood pressure value measured and behavior record data indicating behavior performed by a user and determines, based on the user-related data acquired, an essential hypertension prediction model based on a behavior score indicating the behavior of the user, and a secondary hypertension prediction model, degrees of possibilities of essential hypertension and secondary hypertension.
11. An apparatus according to an aspect of the present invention is an apparatus for determining a degree of possibility of secondary hypertension. The apparatus acquires user-related data including blood pressure value data indicating a blood pressure value measured and behavior record data indicating behavior performed by a user and determines, based on the user-related data acquired, an essential hypertension prediction model based on a behavior score indicating the behavior of the user, and a secondary hypertension prediction model, degrees of possibilities of essential hypertension and secondary hypertension.

### Advantageous Effects of Invention

Use of the present invention makes it possible to determine a degree of possibility of secondary hypertension for a patient whose blood pressure is not lowered even though a behavior change for lowering the blood pressure is made.

### BRIEF DESCRIPTION OF DRAWINGS

FIG. 1 is a configuration diagram of a system according to an embodiment of the present invention.

FIG. 2 is a hardware configuration diagram of a client apparatus and a server according to an embodiment of the present invention.

FIG. 3 is a functional block diagram of a determination server according to an embodiment of the present invention.

FIG. 4 is a functional block diagram of a patient's terminal according to an embodiment of the present invention.

FIG. 5 is a functional block diagram of a doctor's terminal according to an embodiment of the present invention.

FIG. 6 is a flowchart according to an embodiment of the present invention.

FIG. 7 shows an example of user-related data according to an embodiment of the present invention.

FIG. 8A shows an example of values of variables of an essential hypertension prediction model and a secondary hypertension prediction model according to an embodiment of the present invention.

FIG. 8B shows values of variables of an essential hypertension prediction model and a secondary hypertension prediction model calculated from values of FIGS. 7 and 8A according to an embodiment of the present invention.

FIG. 9 is a sequence chart according to an embodiment of the present invention.

FIG. 10 is a sequence chart according to an embodiment of the present invention.

FIG. 11 illustrates an example of a patient's terminal screen according to an embodiment of the present invention.

FIG. 12 illustrates an example of a patient's terminal screen according to an embodiment of the present invention.

FIG. 13 illustrates an example of a patient's terminal screen according to an embodiment of the present invention.

FIG. 14 illustrates an example of a patient's terminal screen according to an embodiment of the present invention.

FIG. 15 illustrates an example of a doctor's terminal screen according to an embodiment of the present invention.

### DESCRIPTION OF EMBODIMENTS

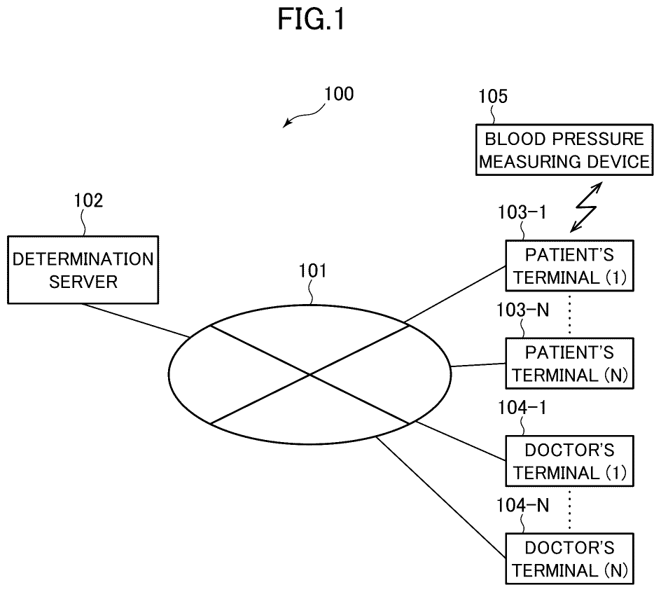

FIG. 1 illustrates an example of a system configuration diagram of the present invention. A system 100 is used for determining a degree of possibility of secondary hypertension and includes a network 101, a determination server 102, a patient's terminal 103, and a doctor's terminal 104. Further, in the present embodiment, it is assumed that a blood pressure measuring device 105 related to blood pressure measurement, which is connected to the patient's terminal 103, is included. For example, the blood pressure measuring device 105 can perform blood pressure measurement according to an instruction from a therapeutic app. Further, for example, the blood pressure measuring device 105 can operate in conjunction with the therapeutic app by being wirelessly connected to the patient's terminal 103.

In the present embodiment, although the therapeutic app (therapeutic application) means an application that improves recognition or behavior related to hypertension of a user and that is used for therapy of hypertension, any application may be used as long as the application is used to improve hypertension of the user. In this specification, a therapeutic app and an app for therapy are used in the same meaning. In addition, in this specification, a user who uses the therapeutic app to improve hypertension is referred to as a user or a patient. In this specification, hypertension includes not only medical hypertension but also a state in which a blood pressure is so high that it is preferable to make an improvement in terms of health. Therapy is not limited to therapy performed under an instruction from a medical service worker but may include therapy performed for health improvement. In addition, in the embodiment of this specification, a doctor is described as an example of a medical service worker, but a medical service worker other than a doctor can similarly implement the present invention.

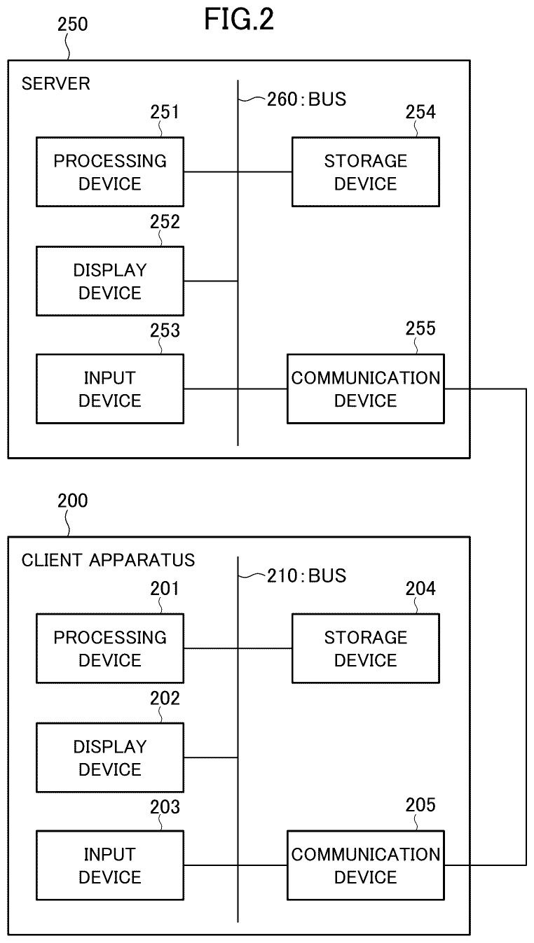

FIG. 2 is a block diagram illustrating a hardware configuration of a client apparatus 200 that can be used as each of the patient's terminal 103 and the doctor's terminal 104 and a server 250 that can be used as the determination server 102 according to an embodiment of the present invention. The client apparatus 200 includes a processing device 201, a display device 202, an input device 203, a storage device 204, and a communication device 205. These constituent devices are connected to each other by a bus 210. Note that an interface is assumed to be interposed between the bus 210 and each constituent device as necessary. In the present embodiment, the client apparatus 200 as the patient's terminal is a smartphone, the doctor's terminal is a terminal used by a doctor, and the client apparatus 200 as the doctor's terminal is a personal computer. However, the client apparatus 200 can be another information processing device such as a tablet computer and a touch pad as long as the client apparatus 200 includes the configuration describe above.

The server 250 also similarly includes a processing device 251, a display device 252, an input device 253, a storage device 254, and a communication device 255. These constituent devices are connected to each other by a bus 260. Note that an interface is assumed to be interposed between the bus 260 and each constituent device as necessary. In the present embodiment, the server 250 is implemented by a computer or another information processing device that operates as a server.

The processing devices 201 and 251 control operations of the whole client apparatus 200 and the whole server 250 and are, for example, CPUs. The processing devices 201 and 251 executes various types of processing by reading and executing programs and data stored in the storage devices 204 and 254. In one example, the processing devices 201 and 251 are composed of a plurality of processors.

The display devices 202 and 252 display application screens and the like for a user (patient or doctor) of the client apparatus 200 and a user (administrator) of the server 250 under control of the processing devices 201 and 251. Each display device can be a liquid crystal display, a display using organic EL, a plasma display, or the like.

The input devices 203 and 253 are user interfaces that receive input from users to the client apparatus 200 and the server 250, and are for example, touch panels, touch pads, keyboards, or mouses. In the present embodiment, the client apparatus 200 as the patient's terminal is a smartphone. Thus, the client apparatus 200 includes a touch panel as the input device 203, the touch panel also functions as the display device 202, and the display device 202 and the input device 203 are integrally configured. The display device 202 and the input device 203 may be in separate forms in which the display device and the input device are disposed at separate positions. Here, the client apparatus 200 as the doctor's terminal and the server 250 are computers and thus are assumed to each include a keyboard and a mouse as the input device, and a liquid crystal display as the display device.

The storage devices 204 and 254 are storage devices provided in typical smartphones and computers, the storage devices including a storage device, a magnetic storage device, or the like including a RAM as a volatile memory and a flash memory such as an eMMC, a UFS, or an SSD as a nonvolatile memory. The storage devices 204 and 254 each can also include an external memory.

For example, when the client apparatus 200 is used as the patient's terminal, a program of the therapeutic app is stored and installed in the storage device 204, so that the client apparatus 200 can function as the patient's terminal capable of executing the therapeutic app. When the client apparatus 200 is used as the doctor's terminal, a program for the doctor's terminal is stored and installed in the storage device 204. In the storage device of the server 250, a server program for determination processing is similarly stored for the determination server. A server program for the therapeutic app includes a function and various types of data for performing information processing so as to appropriately operate the therapeutic app executed in each patient's terminal or each doctor's terminal as a client. Further, a database physically separated from the client apparatus 200 and the server 250 can be used as the storage device.

The communication devices 205 and 255 transmit and/or receive data to and/or from other devices via the network 101 (omitted in FIG. 2). For example, the communication device 205 performs mobile communication and wireless communication via a wireless LAN and is connected to the network 101. The client apparatuses 200 use the respective communication devices 205 and thus perform communication with the server 250 via the network 101. The communication devices 205 and 255 may perform wired communication using an Ethernet (registered trademark name) cable or the like.

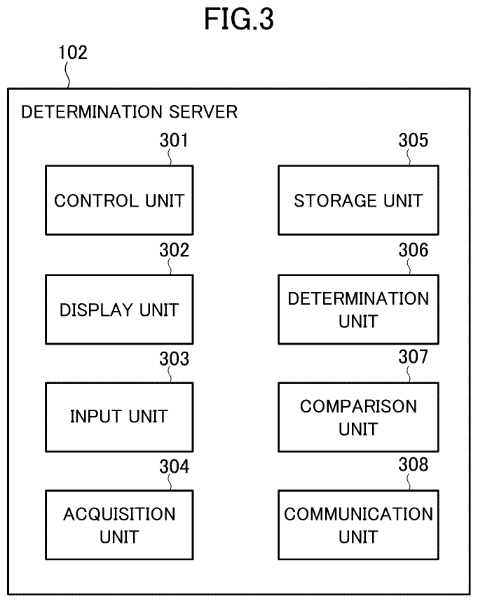

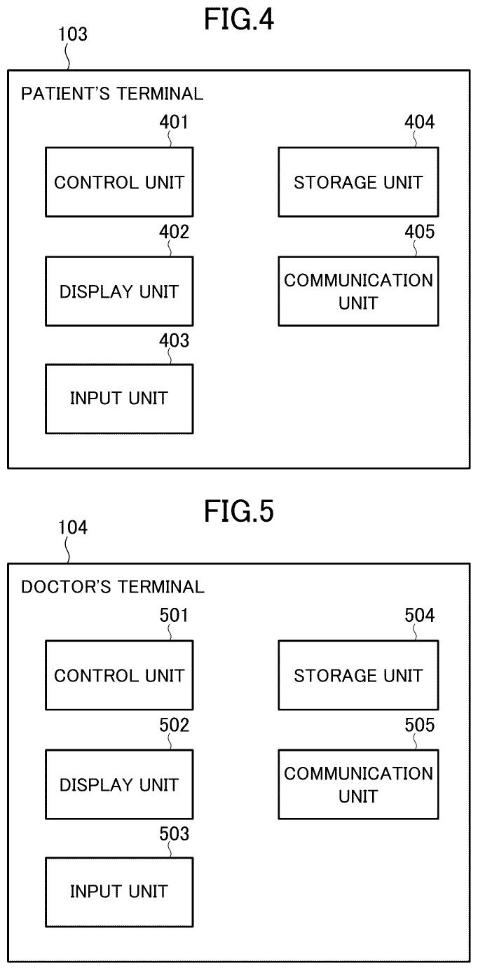

FIGS. 3 to 5 illustrate examples of functional block diagrams of the determination server 102, the patient's terminal 103, and the doctor's terminal 104, respectively, according to an embodiment of the present invention. The determination server 102 includes a control unit 301, a display unit 302, an input unit 303, an acquisition unit 304, a storage unit 305, a determination unit 306, a comparison unit 307, and a communication unit 308. The patient's terminal 103 includes a control unit 401, a display unit 402, an input unit 403, a storage unit 404, a communication unit 405, and a position determination unit 406. The doctor's terminal 104 includes a control unit 501, a display unit 502, an input unit 503, a storage unit 504, and a communication unit 505.

In the present embodiment, these functions are implemented by the processing devices 201 and 251 executing the determination server program, the therapeutic app for the patient's terminal, the therapeutic app for the doctor's terminal, and the like stored in the storage devices 204 and 254. Since various functions are implemented by program reading, one part (function) may be partly or wholly owned by another part. These functions may be implemented by hardware by constituting an electronic circuit or the like for implementing a part or the whole of each function. These functional units do not need to be implemented on one physical information processing device. These functional units may be implemented on a plurality of information processing devices, and each storage unit may be wholly or partly implemented as a database. Any of the functional units may be implemented on another device.

The control units 301, 401, and 501 perform control processing when executing information processing for therapy by the therapeutic app of the present embodiment, processing of determining the degrees of possibilities of essential hypertension and secondary hypertension in the determination unit 306, and processing of comparing the degrees of possibilities of essential hypertension and secondary hypertension in the comparison unit 307. The storage units 305, 404, and 504 store data necessary for these processing operations and data acquired through these processing operations. The display units 302, 402, and 502 are configured of the display devices 202 and 252 and present information to a user of each device. Note that in addition to or instead of the display units, a presentation unit that presents information by voice, touch, or the like may be provided. The input units 303, 403, and 503 are configured of the input devices 203 and 253 and receive input from users of the respective devices. In the present embodiment, a touch detection function that a smartphone including a touch panel typically has can be used in the patient's terminal 103. The communication units 308, 405, and 505 are configured of the communication devices 205 and 255 and transmit and/or receive information to and/or from another server, patient's terminal, doctor's terminal, or the like.

The acquisition unit 304 of the determination server 102 is implemented by the communication device 255 and acquires user-related data of a patient who undergoes therapy through the use of the therapeutic app. The storage unit 404 stores the acquired user-related data in association with information (user identification information) that can identify the patient from whom the user-related data has been acquired. For example, the acquired user-related data is stored in association with app identification information of the therapeutic app used by the patient from whom the user-related data has been acquired.

Here, the user-related data will be described. The user-related data includes blood pressure value data and behavior record data. The blood pressure value data is data indicating a measured blood pressure value and may include at least one of a systolic blood pressure value and a diastolic blood pressure value. The blood pressure value data can be measured by the blood pressure measuring device 105 or the like. The behavior record data includes data indicating behavior performed by the patient. Examples of the behavior record data can include the amount of exercise, the weight, and the amount of salt intake of the user. The user-related data includes the blood pressure value data and the behavior record data of a plurality of days, and each piece of the blood pressure value data and each piece of the behavior record data are stored in association with the date to which each piece of the data relates. The blood pressure value data can be associated with the date on which the blood pressure has been measured, and the behavior record data can be associated with the date on which the patient has performed behavior indicated by the behavior record data.

The behavior record data may include subjective data and objective data. Examples of the subjective data of the behavior record data may include data obtained by the patient himself/herself subjectively evaluating the patient's effort to do exercise or reduce salt intake. Examples of the objective data of the behavior record data may include temperature data and weight data. The temperature data may be acquirable temperature data at a representative location at a representative time of a date associated with the user-related data. The temperature of a region where the patient has performed behavior may be input or may be acquired from a weather information server that manages the temperature data of each region. Position information of the region where the patient has performed behavior may be acquired from the patient's terminal 103. Otherwise, the region where the patient has performed behavior may be input by the patient or may be a region including the address of the patient included in profile information or the like of the patient. Regarding the patient's effort to do exercise or reduce salt intake, objective data may be used in addition to or instead of data obtained by subjective evaluation by the patient himself/herself. For example, the number of steps of the patient in one day measured by a pedometer or the daily calorie consumption of the patient estimated using a wearable device may be used as the data indicating the patient's effort to do exercise. The measured amount of salt taken by the patient in one day may be used as the data indicating the effort to reduce salt intake.

The patient's terminal 103 can receive the user-related data input by the patient via the installed therapeutic app. The patient can input the user-related data of a single day or input the user-related data of a plurality of days daily or collectively. The patient's terminal 103 transmits the user-related data input by the patient to the determination server 102. The user-related data can be transmitted from the patient's terminal 103 to the determination server 102 at a predetermined timing. For example, the transmission may be performed at a predetermined time every day, may be performed once a week, or may be performed at a timing of executing the processing of determining the degree of possibility of secondary hypertension.

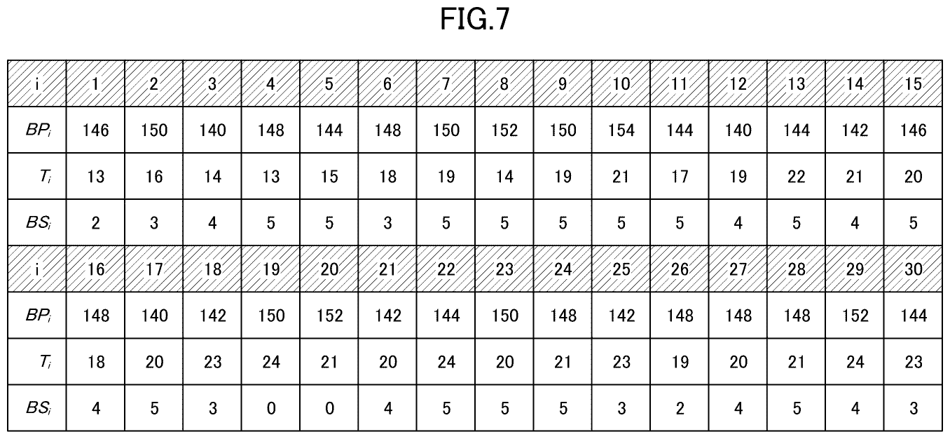

Regarding the user-related data, FIG. 7 shows an example of the user-related data according to an embodiment of the present invention. In the example shown in FIG. 7, a systolic blood pressure value BPᵢ, a temperature Ti, and a behavior score BSᵢ of an i-th day are shown, and the example is based on the user-related data obtained by the patient inputting these values for 30 days (i=1 to 30). For example, on the first day (i=1), a systolic blood pressure value is 146, a temperature is 13, and a behavior score is 2. Here, the behavior score is based on the behavior record data included in the user-related data. For example, the behavior score can be determined based on scores of “effort to reduce salt intake” and “effort to do exercise” subjectively scored by the patient on a screen 1200 of FIG. 12 described below.

The functional block diagram of the determination server 102 in FIG. 3 will be described again. The determination unit 306 determines the degrees of possibilities of essential hypertension and secondary hypertension based on the user-related data acquired by the acquisition unit 304. As an embodiment, the degrees of possibilities of essential hypertension and secondary hypertension are determined based on the user-related data acquired by the acquisition unit 304, an essential hypertension prediction model, and a secondary hypertension prediction model. The degrees of possibilities of essential hypertension and secondary hypertension may be determined based on the user-related data acquired by the acquisition unit 304, a likelihood when hypertension is assumed to be essential hypertension, and likelihood when hypertension is assumed to be secondary hypertension. The likelihood when hypertension is assumed to be essential hypertension and the likelihood when hypertension is assumed to be secondary hypertension are calculated based on the essential hypertension prediction model and the secondary hypertension prediction model.

The comparison unit 307 compares the degrees of possibilities of essential hypertension and secondary hypertension determined by the determination unit 306 to determine the degree of possibility of secondary hypertension compared with the possibility of essential hypertension. Although the comparison unit 307 is included in the present embodiment, the comparison unit 307 does not need to be included in one modification example.

The display unit 402 of the patient's terminal 103 or the display unit 502 of the doctor's terminal 104 displays information indicating the degree of possibility of secondary hypertension determined in comparison with the possibility of essential hypertension. When the comparison unit 307 is not included, information indicating the degrees of possibilities of essential hypertension and secondary hypertension determined based on the likelihood when hypertension is assumed to be essential hypertension and the likelihood when hypertension is assumed to be secondary hypertension may be displayed.

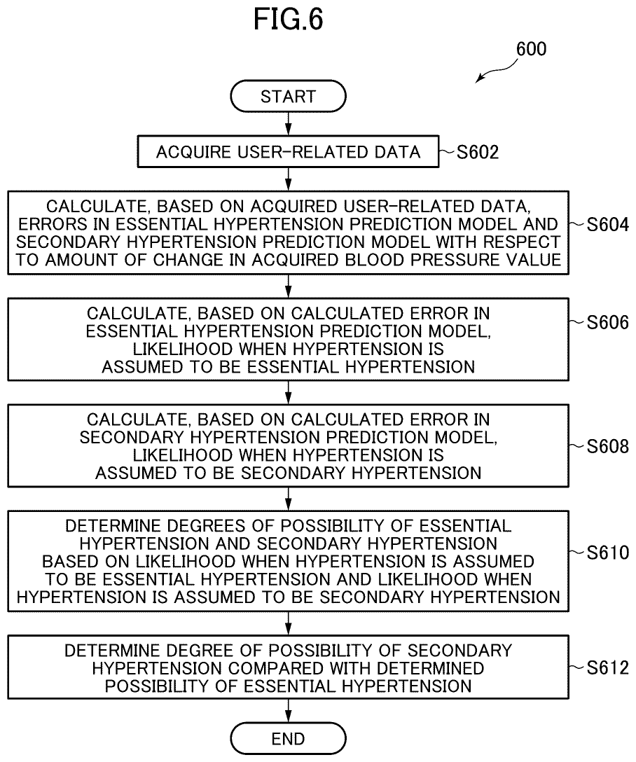

FIG. 6 illustrates a flowchart performed by the system 100 for determining the degrees of possibilities of essential hypertension and secondary hypertension according to an embodiment. A hypertensive patient starts therapy using the therapeutic app, measures a blood pressure, and inputs user-related data to the patient's terminal via the therapeutic app. In step S602, the user-related data input by the patient in this way is acquired.

In step S604, errors of the essential hypertension prediction model and the secondary hypertension prediction model with respect to the amount of change in acquired blood pressure value are calculated based on the user-related data acquired in step S602. For example, the amount of change in blood pressure value may be the amount of change from a blood pressure value of a certain day to a blood pressure value of another certain day when a systolic blood pressure value and a diastolic blood pressure value are measured for 30 days, for example, the amount of change from a systolic blood pressure value of the first day to a systolic blood pressure value of the 30th day, or the amount of change from an average systolic blood pressure value of a plurality of days to an average systolic blood pressure value of another plurality of days, for example, the amount of change from an average systolic blood pressure value of four days from the first day to fourth day to an average systolic blood pressure value of four days from the 27th day to 30th day. The blood pressure value here is not limited to an average blood pressure value, and may be a median value or a value calculated by another statistical method from blood pressure values in a predetermined time period.

The essential hypertension prediction model and the secondary hypertension prediction model will be described. When the patient has essential hypertension, an error of the essential hypertension prediction model with respect to the amount of change in acquired blood pressure value is small, and an error of the secondary hypertension prediction model with respect to the amount of change in acquired blood pressure value is large. On the other hand, when the patient has secondary hypertension, an error of the essential hypertension prediction model with respect to the amount of change in acquired blood pressure value is large, and an error of the secondary hypertension prediction model with respect to the amount of change in acquired blood pressure value is small.

Since essential hypertension is considered to be affected by behavior of the patient such as meals and lifestyle habits, the essential hypertension prediction model of the present embodiment is a model based on the behavior score, more specifically, a model including a term related to the behavior score. Since secondary hypertension is based on a disease and is considered to be hardly affected by behavior of the patient, the secondary hypertension prediction model of the present embodiment is a model not based on the behavior score, more specifically, a model not including a term related to the behavior score. Secondary hypertension may be based on the behavior score when considered to be affected by certain behavior of the patient. In this case, a coefficient given to the behavior score can be made smaller than that of the essential hypertension prediction model. Alternatively, the behavior score may be limited to a behavior score related to behavior considered to affect secondary hypertension.

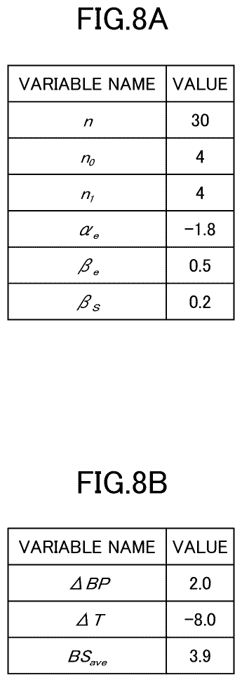

The essential hypertension prediction model of an embodiment is indicated by Equation 1 below. Each term of Equation 1 will be described below. ΔBPₑ represents the amount of change in acquired blood pressure value, and in one example, is the amount of change in average systolic blood pressure value from an average systolic blood pressure value of four days from the first day to fourth day to an average systolic blood pressure value of four days from the 27th day to 30th day shown in FIG. 7, and ΔBPₑ is 2.0 as shown in FIG. 8B. αₑ represents a regression coefficient of an average behavior score value in the essential hypertension prediction model and, in one example, is −1.8 as shown in FIG. 8B. BSₐᵥₑ represents an average value of behavior scores from the first day to the n-th day and, in one example, is calculated by Equation 2 below, and an average value of behavior scores from the first day to the 30th day in FIG. 7 is 3.9 as shown in FIG. 8B. βₑ represents a regression coefficient of a temperature difference in the essential hypertension prediction model and, in one example, is 0.5 as shown in FIG. 8B.

ΔT represents the amount of change in acquired temperature. Here, for example, when temperatures are acquired for 30 days, the amount of change in temperature may be the amount of change from a temperature of a certain day to a temperature of another certain day, for example, the amount of change from a temperature of the first day to a temperature of the 30th day. Moreover, when no days from an x-th day to a (x+n₀−1)-th day are defined as a start period and n₁ days from a (y−n₁+1)-th day to a y-th day are defined as an end period, the amount of change in temperature may be the amount of change in average temperature from an average temperature of no days in the start period to an average temperature of n₁ days in the end period. In this case, ΔT can be obtained by Equation 3. For example, when four days from the first day to the fourth day are defined as the start period and four days from the 27th day to the 30th day are defined as the end period, the amount of change in average temperature from an average temperature of four days from the first day to the fourth day in the start period to an average temperature of four days from the 27th day to the 30th day in the end period may be used. In the example of FIG. 7, ΔT is −0.8 as shown in FIG. 8B. εₑ represents an error of the sum of αₑBSₐᵥₑ and βₑΔT with respect to the amount ΔBPₑ of change in acquired blood pressure value and is 13.02 in one example.

$$Δ {BP}_{e} = {α}_{e} B {S}_{ave} + {β}_{ε} Δ T + {ε}_{e} \qquad [ Math . 1 ]$$

Here, Equations 2 and 3 will be described. The average value of the behavior scores from the first day to the n-th day is obtained by Equation 2 below. BSₖ represents a behavior score of a k-th day. In one example, n is 30.

$$B {S}_{ave} = \frac{1}{n} {\sum }_{k = 1}^{n} B {S}_{k} \qquad [ Math . 2 ]$$

The amount of change in temperature from a temperature of the first day to a temperature of the n-th day is obtained by Equation 3 below. n₀ represents the number of samples used for obtaining the average temperature for no days from the x-th day to the (x+n₀−1)-th day of the start period. In one example, the average temperature of the start period is obtained from the temperatures of the first day to the fourth day in FIG. 7, and n₀ is 4 as shown in FIG. 8A. n₁ represents the number of samples used for obtaining the average temperature of n₁ days from the (y−n₁+1) day to the y-th day in the end period. In one example, the average temperature of the end period is obtained from the temperatures of the 27th day to the 30th day in FIG. 7, and n₁ is 4 as shown in FIG. 8A. Thus, ΔT is obtained from Equation 4 below in one example and is −8.0 as shown in FIG. 8B.

$$\begin{matrix}Δ T = \frac{1}{{n}_{0}} {\sum }_{k = 1}^{{n}_{0}} & {T}_{k} - \frac{1}{{n}_{1}} {\sum }_{k = 1}^{{n}_{1}} & {T}_{n - k + 1}\end{matrix} \qquad [ Math . 3 ]$$

Next, the secondary hypertension prediction model according to an embodiment is indicated by Equation 4 below. Each term of Equation 4 will be described below. ΔBPₛ represents the amount of change in acquired blood pressure value and, in one example, is the amount of change in average systolic blood pressure value from an average systolic blood pressure value of four days from the first day to fourth day to an average systolic blood pressure value of four days from the 27th day to 30th day shown in FIG. 7 and is 2.0 as shown in FIG. 8B. βₛ represents a regression coefficient of a temperature difference in the secondary hypertension prediction model and, in one example, is 0.2 as shown in FIG. 8A. ΔT represents the amount of change in average temperature from an average temperature of a start period to an average temperature of an end period, is obtained by Equation 3 above in one example, and is −8.0 as shown in FIG. 8B. εₛ represents an error of βₛΔT with respect to the amount ΔBPₛ of change in acquired blood pressure value and is 3.6 in one example.

$$Δ {BP}_{s} = {β}_{s} Δ T + {ε}_{s} \qquad [ Math . 4 ]$$

In step S606, based on the error in the essential hypertension prediction model with respect to the amount of change in acquired blood pressure value, which has been calculated in step S604, likelihood when hypertension is assumed to be essential hypertension is calculated. The degree of possibility of essential hypertension is indicated by, for example, the likelihood when hypertension is assumed to be essential hypertension based on the calculated error εₑ in one embodiment. In one example, assuming that the error εₑ follows a normal distribution N (0, σₑ²) of the essential hypertension prediction model and that the standard deviation σₑ is equal to 6, the likelihood when hypertension is assumed to be essential hypertension is obtained by Equation 5 below and is 0.0178.

$$P ( \vert {ε}_{e} \vert \leq 13.02 ) = p ( z \leq 2.17 ) = 0.0178 \qquad [ Math . 5 ]$$

In step S608, based on the error in the secondary hypertension prediction model with respect to the amount of change in acquired blood pressure value, which has been calculated in step S604, likelihood when hypertension is assumed to be secondary hypertension is calculated. The degree of possibility of secondary hypertension is indicated by, for example, the likelihood when hypertension is assumed to be secondary hypertension based on the calculated error εₛ in one embodiment. In one example, assuming that the error εₛ follows a normal distribution N (0, σₛ²) of the secondary hypertension prediction model and that the standard deviation σₛ is equal to 6, the likelihood when hypertension is assumed to be secondary hypertension is obtained by Equation 6 below and is 0.2742.

$$P ( \vert {ε}_{s} \vert \leq 3.6 ) = p ( z \leq 0.6 ) = 0.2742 \qquad [ Math . 6 ]$$

In step S610, the degrees of possibilities of essential hypertension and secondary hypertension are determined based on the likelihood when hypertension is assumed to be essential hypertension and the likelihood when hypertension is assumed to be secondary hypertension calculated in steps S606 and S608. Here, the degrees of possibilities of essential hypertension and secondary hypertension are the calculated likelihood itself when hypertension is assumed to be essential hypertension and the calculated likelihood itself when hypertension is assumed to be secondary hypertension, respectively.

In step S612, the degree of possibility of secondary hypertension compared to the possibility of essential hypertension determined in step S610 is determined. Here, the degree of possibility of secondary hypertension compared with the possibility of essential hypertension is determined by calculating a likelihood ratio of the likelihood when hypertension is assumed to be secondary hypertension with respect to the likelihood when hypertension is assumed to be essential hypertension determined in step S610 (hereinafter referred to as the likelihood ratio of secondary hypertension).

The likelihood ratio of secondary hypertension is obtained by dividing the likelihood when hypertension is assumed to be secondary hypertension, which has been calculated in step S608, by the likelihood when hypertension is assumed to be essential hypertension, which has been calculated in step S606. For example, in one example, the likelihood ratio of secondary hypertension is approximately 15.4, which is obtained by dividing 0.2742, which is the likelihood when hypertension is assumed to be secondary hypertension, by 0.0178, which is the likelihood when hypertension is assumed to be essential hypertension. From the value of this likelihood ratio, the degree of possibility of secondary hypertension can be determined to be 15.4 times the possibility of essential hypertension. The degree of possibility of essential hypertension compared with the possibility of secondary hypertension may be indicated by a likelihood ratio of the likelihood when hypertension is assumed to be essential hypertension with respect to the likelihood when hypertension is assumed to be secondary hypertension (hereinafter referred to as a likelihood ratio of essential hypertension). The likelihood ratio of essential hypertension may be obtained by dividing the likelihood when hypertension is assumed to be essential hypertension by the likelihood when hypertension is assumed to be secondary hypertension.

The degrees of possibilities of essential hypertension and secondary hypertension described above may be determined using the essential hypertension prediction model and the secondary hypertension prediction model based on a machine learning model.

The determination server 102 may present information indicating the degree of possibility of secondary hypertension determined as described above to the doctor via the doctor's terminal 104 or to the patient via the patient's terminal 103.

In the present embodiment, the comparison processing in step S612 is included, but in other embodiments, the comparison processing does not need to be included. In this case, the patient's terminal 103 and the doctor's terminal 104 can present the determined degrees of possibilities of essential hypertension and secondary hypertension. The degrees of possibilities of essential hypertension and secondary hypertension may be the likelihood itself when hypertension is assumed to be essential hypertension and the likelihood itself when hypertension is assumed to be secondary hypertension calculated by Equations 5 and 6 or may be numerical values obtained by executing predetermined calculation processing on the likelihood when hypertension is assumed to be essential hypertension and the likelihood when hypertension is assumed to be secondary hypertension. The likelihoods or the obtained numerical values may be values having no range or values having ranges. In addition, indexes determined based on the likelihood when hypertension is assumed to be essential hypertension and the likelihood when hypertension is assumed to be secondary hypertension may be used. For example, when the calculated likelihood when hypertension is assumed to be essential hypertension and the calculated likelihood when hypertension is assumed to be secondary hypertension are equal to or greater than preset threshold values, indexes indicating that the degrees of possibilities are high can be presented, and when the likelihoods are less than the threshold values, indexes indicating that the degrees of possibilities are low can be presented.

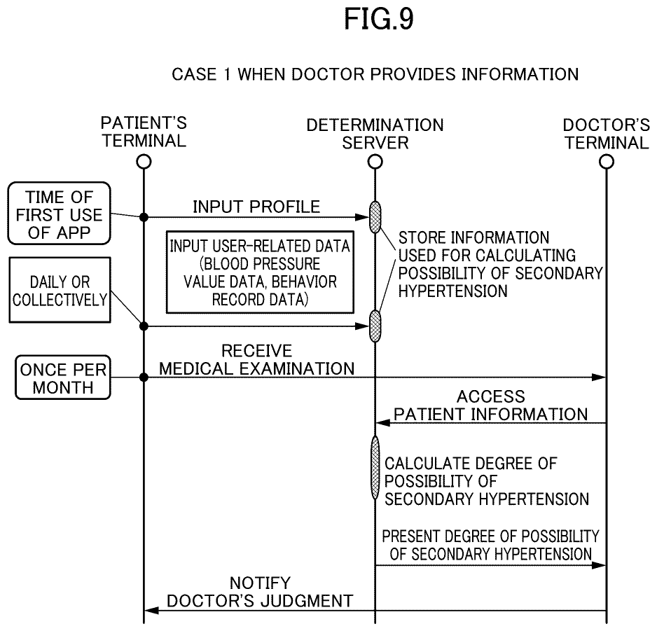

FIG. 9 illustrates a sequence chart for presenting, upon input of request operation to the doctor's terminal, information indicating the degree of possibility of secondary hypertension determined as described above on the doctor's terminal 104. A patient who is prescribed with the therapeutic app installs the therapeutic app in the patient's terminal 103 and inputs user information such as the patient's profile to the patient's terminal 103 via the therapeutic app. The determination server 102 acquires the user information and stores the user information in association with user identification information. The user identification information may be a user name of the patient or may be app identification information of the therapeutic app used by the patient. Next, the patient inputs user-related data indicating blood pressure value data and behavior record data to the patient's terminal 103 via the therapeutic app. The patient may daily input the user-related data or may collectively input the user-related data of a plurality of days.

The patient's terminal 103 transmits the user-related data input by the patient to the determination server 102, and the determination server 102 receives and acquires, from the patient's terminal 103, the user-related data of the patient who receives therapy by using the therapeutic app. The determination server 102 stores the acquired user-related data in association with the user identification information of the patient from whom the data has been acquired. The acquired user-related data may be stored in association with the acquired profile information or the like of the patient or may be stored in association with the acquired app identification information of the therapeutic app used by the patient.

Next, the patient visits a medical institution, for example, 30 days after being prescribed with the therapeutic app. Upon input from the doctor, the doctor's terminal 104 transmits a request of presenting the patient information including the user-related data stored in the determination server 102. The determination server 102 determines the degree of possibility of secondary hypertension based on the user-related data associated with the stored user identification information regarding the patient who receives the medical examination. The determination server 102 transmits the patient information including information indicating the determined degree of possibility of secondary hypertension to the doctor's terminal 104. The doctor's terminal 104 presents, to the doctor, the patient information including the information indicating the determined degree of possibility of secondary hypertension acquired from the determination server 102.

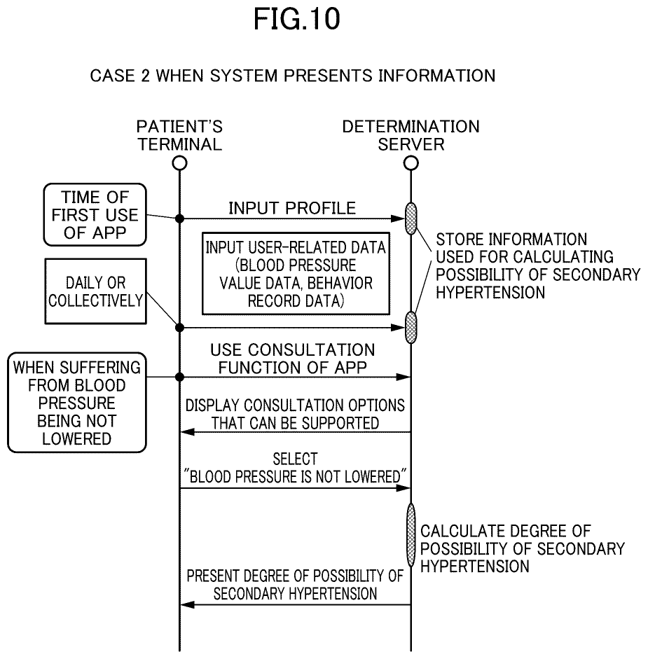

FIG. 10 illustrates a sequence chart for presenting, upon input of request operation to the patient's terminal, information indicating the degree of possibility of secondary hypertension determined as described above on the patient's terminal 103. A patient who is prescribed with the therapeutic app inputs user information such as the patient's profile to the patient's terminal 103 via the therapeutic app installed in the patient's terminal 103. The determination server 102 acquires the user information and stores the user information in association with user identification information. The user identification information may be app identification information of the therapeutic app used by the patient. Next, the patient inputs user-related data indicating blood pressure value data and behavior record data to the patient's terminal 103 via the therapeutic app. The patient may daily input the user-related data or may collectively input the user-related data of a plurality of days. The determination server 102 acquires, from the patient's terminal 103, the user-related data of the patient who receives therapy by using the therapeutic app. The determination server 102 stores the acquired user-related data in association with the user identification information. The acquired user-related data may be stored in association with the acquired profile or the like of the patient or may be stored in association with the acquired app identification information of the therapeutic app used by the patient.

Next, the patient executes a consultation function of the therapeutic app on the patient's terminal 103, for example, when suffering from the blood pressure not being lowered. The patient's terminal 103 transmits information indicating the execution of the consultation function to the server, and the determination server 102 presents, to the patient via the patient's terminal 103, consultation options that can be supported for the patient. The patient selects the option “the blood pressure is not lowered” from the presented consultation options that can be supported. The determination server 102 determines the degree of possibility of secondary hypertension based on the user-related data associated with the stored user identification information for the patient. The patient's terminal 103 presents, to the patient, data indicating the determined degree of possibility of secondary hypertension acquired from the determination server 102 and information indicating a proposal to consult with a doctor.

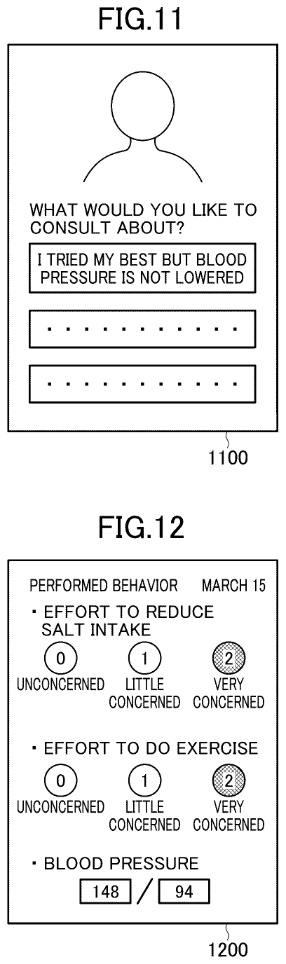

FIGS. 11 to 14 each illustrate an example of a screen displayed on the patient's terminal installed with the therapeutic app according to an embodiment of the present invention.

FIG. 11 is an example of a screen displayed when the patient uses the consultation function of the therapeutic app. On a screen 1100, the question “What would you like to consult about?” is displayed. In addition, conceivable items that the patient would like to consult about are displayed on the screen 1100. For example, the item “I tried my best but the blood pressure is not lowered” is displayed. The number of conceivable items that the patient would like to consult about may be one or more.

FIG. 12 illustrates an example of a screen displayed when the patient records user-related data in the therapeutic app. On the screen 1200, the items “date”, “effort to reduce salt intake” and “effort to do exercise” as performed behavior, and “blood pressure” are displayed. Regarding the item “effort to reduce salt intake” on the screen 1200, the patient can subjectively score his or her effort to reduce salt intake. For example, the patient selects from the options of “0 point: Unconcerned”, “1 point: Little concerned”, and “2 points: Very concerned”. Regarding the item “effort to do exercise” on the screen 1200, the patient can subjectively score his or her effort to do exercise. For example, the patient selects from the options of “0 point: Unconcerned”, “1 point: Little concerned”, and “2 points: Very concerned”. Regarding the item “blood pressure” on the screen 1200, a systolic blood pressure value and a diastolic blood pressure value are input. As the systolic blood pressure value and the diastolic blood pressure value, blood pressure values measured by the blood pressure measuring device may be input by the patient or may be input by the patient's terminal connected to the blood pressure measuring device.

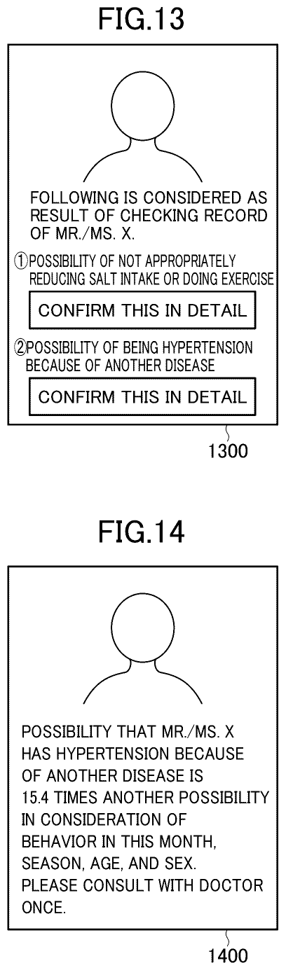

FIG. 13 is an example of a screen displayed when the patient selects the item “I tried my best but the blood pressure is not lowered” on the screen 1100 of FIG. 11. On a screen 1300, the message “The following is considered as a result of checking the record of Mr./Ms. X (for example, the name of the patient)” is displayed. In addition, on the screen 1300, consultation options that can be supported are displayed based on the user-related data input on the screen 1100 of FIG. 11. For example, the items “1. Possibility of not appropriately reducing salt intake or doing exercise” and “2. Possibility of being hypertension because of another disease” are displayed. In addition, options for confirming the respective items in detail are displayed. For example, the message “confirm this in detail” is displayed.

FIG. 14 is an example of a screen displayed when the patient selects detailed confirmation of the item “2. Possibility of being hypertension because of another disease” in FIG. 13. A screen 1400 displays information indicating the degree of possibility of secondary hypertension determined as described above. In one embodiment, the screen 1400 displays information indicating the degree of possibility of secondary hypertension determined in comparison with the possibility of essential hypertension. As the degree of possibility of secondary hypertension determined in comparison with the possibility of essential hypertension, for example, the message “The possibility that Mr./Ms. X (for example, the name of the patient) has hypertension because of another disease is 15.4 times another possibility in consideration of behavior of this month, the season, the age, and the sex” is displayed. In addition, a message for prompting the patient to receive a medical examination is displayed. For example, the message “Please consult with a doctor once” is displayed.

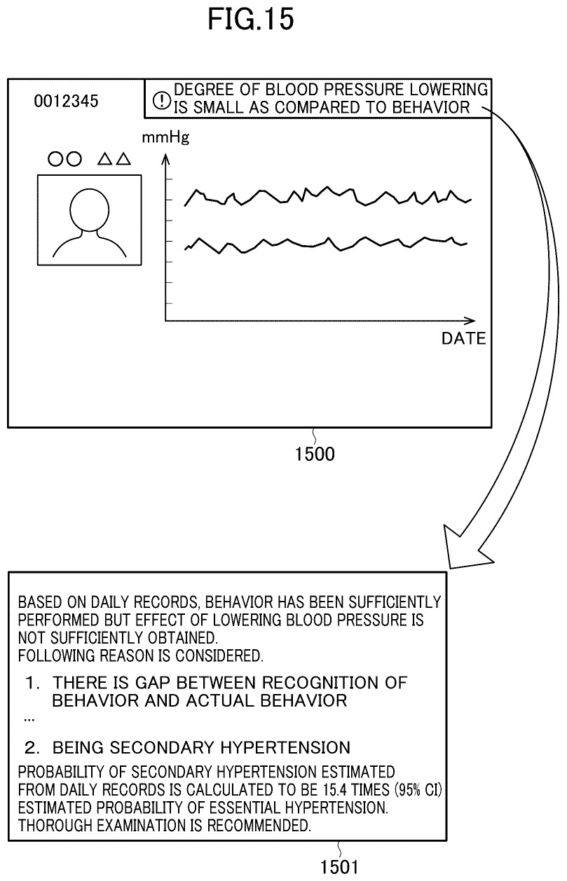

FIG. 15 illustrates an example of a screen displayed on the doctor's terminal 104 installed with a program for the doctor's terminal according to an embodiment of the present invention. A screen 1500 displays information about a patient. As the information about the patient, for example, identification information assigned to the patient and a systolic blood pressure value and a diastolic blood pressure value in a line graph in which the horizontal axis represents the date and the vertical axis represents the blood pressure values are displayed. In addition, a message for giving a warning to the doctor is displayed on the screen 1500. For example, the message “The degree of blood pressure lowering is small as compared to behavior” is displayed. The message may be displayed, for example, when the degree of possibility of secondary hypertension determined as described above is greater than a preset threshold value. When the message is selected, a screen 1501 is displayed. The screen 1501 displays the degree of possibility of secondary hypertension determined as described above. In one embodiment, the screen 1501 displays the degree of possibility of secondary hypertension determined in comparison with the possibility of essential hypertension. As the degree of possibility of secondary hypertension determined in comparison with the possibility of essential hypertension, for example, the following message is displayed: “ . . . 2. Being secondary hypertension The probability of secondary hypertension estimated from the daily records is calculated as 15.4 times (95% confidence interval (CI)) the estimated probability of essential hypertension. A thorough examination is recommended”.

The message for prompting the patient to receive a medical examination in FIG. 14 or the message for giving a warning to the doctor in FIG. 15 can prompt the patient to receive a thorough examination. If the patient actually has secondary hypertension, this may help identify a disease causing secondary hypertension.

In the processing or operations described above, the processing or operations can be modified freely as long as there is no occurrence of contradiction in the processing or operations such as using data that is not yet supposed to be used in a corresponding step. In addition, each example described above is exemplified for describing the present invention, and the present invention is not limited to these examples. The present invention may be implemented in various forms without departing from the scope thereof.

### REFERENCE SIGNS LIST

100: system, 101: network, 102: determination server, 103: patient's terminal, 104: doctor's terminal, 105: blood pressure measuring device, 200: client apparatus, 201: processing device, 202: display device, 203: input device, 204: storage device, 205: communication device, 210: bus, 250: server, 251: processing device, 252: display device, 253: input device, 254: storage device, 255: communication device, 260: bus,
301: control unit, 302: display unit, 303: input unit, 304: acquisition unit, 305: storage unit, 306: determination unit, 307: comparison unit, 308: communication unit,
401: control unit, 402: display unit, 403: input unit, 404: storage unit, 405: communication unit
501: control unit, 502: display unit, 503: input unit, 504: storage unit, 505: communication unit
1100: screen, 1200: screen, 1300: screen, 1400: screen, 1500: screen, 1501: screen

What is claimed is:

1. A method for determining a degree of possibility of secondary hypertension, the method comprising
causing one or more computers to execute
a step of acquiring user-related data including blood pressure value data indicating a blood pressure value measured and behavior record data indicating behavior performed by a user and
a step of determining, based on the user-related data acquired, an essential hypertension prediction model based on a behavior score indicating the behavior of the user, and a secondary hypertension prediction model, degrees of possibilities of essential hypertension and secondary hypertension.

2. The method according to claim 1, wherein
the step of determining degrees of possibilities of essential hypertension and secondary hypertension includes
a step of calculating, based on the user-related data acquired, an error term in the essential hypertension prediction model and an error term in the secondary hypertension prediction model,
a step of calculating, based on the calculated error term in the essential hypertension prediction model, likelihood when hypertension is assumed to be essential hypertension,
a step of calculating, based on the calculated error term in the secondary hypertension prediction model, likelihood when hypertension is assumed to be secondary hypertension, and
a step of determining, based on the calculated likelihood when hypertension is assumed to be essential hypertension and the calculated likelihood when hypertension is assumed to be secondary hypertension, the degrees of possibilities of essential hypertension and secondary hypertension.

3. The method according to claim 1, comprising
further causing the one or more computers to execute a step of determining a degree of possibility of secondary hypertension compared with the determined possibility of essential hypertension.

4. The method according to claim 3, further comprising
a step of presenting, upon input of request operation to a terminal of the user or a medical service worker, information indicating the degree of possibility of secondary hypertension compared with the determined possibility of essential hypertension on the terminal.

5. The method according to claim 1, wherein
the essential hypertension prediction model includes a term related to the behavior score, and
the secondary hypertension prediction model includes no term related to the behavior score.

6. The method according to claim 1, wherein
the behavior record data includes at least one of an amount of exercise, a weight, and an amount of salt intake of the user.

7. The method according to claim 1, wherein
the user-related data is acquired in a predetermined time period and includes two or more pieces of the blood pressure value data and two or more pieces of the behavior record data.

8. The method according to claim 1, wherein
the user-related data includes information indicating the blood pressure value and the behavior performed by the user and is transmitted by a terminal of the user, the information being input by the user to the terminal of the user.

9. A non-transitory computer-readable computer medium storing a computer program for causing one or more computers to execute the method according to claim 1.

10. A system for determining a degree of possibility of secondary hypertension, wherein
the system
acquires user-related data including blood pressure value data indicating a blood pressure value measured and behavior record data indicating behavior performed by a user and
determines, based on the user-related data acquired, an essential hypertension prediction model based on a behavior score indicating the behavior of the user, and a secondary hypertension prediction model, degrees of possibilities of essential hypertension and secondary hypertension.

11. An apparatus for determining a degree of possibility of secondary hypertension, wherein
the apparatus
acquires user-related data including blood pressure value data indicating a blood pressure value measured and behavior record data indicating behavior performed by a user and
determines, based on the user-related data acquired, an essential hypertension prediction model based on a behavior score indicating the behavior of the user, and a secondary hypertension prediction model, degrees of possibilities of essential hypertension and secondary hypertension.
